(19) United States
(12) Patent Application Publication
(10) Pub. No.: US 2013/0281949 A1
(43) Pub. Date: Oct. 24, 2013

(54) POTTY TRAINING LINERS

(71) Applicant: Lori Lee Periman, Austin, TX (US)

(72) Inventor: Lori Lee Periman, Austin, TX (US)

(21) Appl. No.: 13/923,514

(22) Filed: Jun. 21, 2013

Related U.S. Application Data
(63) Continuation-in-part of application No. 12/803,006, filed on Jun. 16, 2010.
Application No. 12/803,006 is a continuation-in-part of application No. 12/285,586, filed on Oct. 9, 2008.
(60) Provisional application No. 60/998,825, filed on Oct. 12, 2007.

(51) Int. Cl. A61L 15/20; A61L 15/56

(52) U.S. Cl. CPC A61L 15/20; A61L 15/56
USPC 604/361

(57) ABSTRACT

A potty training apparatus having a hydrophobic bottom layer and a core disposed on the hydrophobic bottom layer. The potty training apparatus further having a top layer including an acquisition layer disposed above the core and a hydrophilic sensory strip disposed on the top layer. Also, a method of potty training a user including releasing a fluid into a potty training liner, wherein the potty training liner has a sensory section. The method further includes contacting the wet sensory section against the user and producing a sensory signal to the user.

### CROSS-REFERENCE TO RELATED APPLICATIONS

This application claims the benefit, pursuant to 35 U.S.C. §120, as a continuation-in-part application of U.S. application Ser. No. 12/803,006, filed Jun. 16, 2010, which is a continuation-in-part of U.S. application Ser. No. 12/285,586, filed Oct. 9, 2008, and U.S. application Ser. No. 12/285,586, filed Oct. 9, 2008, which claimed priority to U.S. Provisional Application Ser. No. 60/998,825, filed Oct. 12, 2007. These applications are incorporated by reference in their entirety.

### BACKGROUND OF THE INVENTION

The present disclosure relates generally to devices that function as an aid to children transitioning from diapers to underwear. Pull-on type diapers are presently used for potty training, as they allow a child to pull them down, like underwear, to go to the bathroom.

Generally, pull on training pants have properties that make them similar to diapers, which a potty training child has been wearing their entire life. Both diapers and training pants are highly absorbent and wick fluid away from the body quickly and efficiently. Modern diapers and training pants often wick fluid away from the body so quickly and keep the user feeling comfortable and dry after an accident that the user does not know they have had an incident. And because training pants are so similar to diapers, it is difficult for a potty training child to differentiate the potty training stage and potty training is delayed.

Prior to the advent of disposable diapers and training pants, the average age for potty training children was between 18 and 24 months. With the advent of disposable diapers and training pants, the average age of potty training children has crept up to between 3 and 4 years.

### BRIEF SUMMARY OF THE INVENTION

According to one aspect of one or more embodiments of the present invention, a potty training apparatus having a hydrophobic bottom layer and a core disposed on the hydrophobic bottom layer. The potty training apparatus further having a top layer including an acquisition layer disposed above the core and a hydrophilic sensory strip disposed on the top layer.

According to another aspect of one or more embodiments of the present invention, a method of potty training a user including releasing a fluid into a potty training liner, wherein the potty training liner has a sensory section. The method further includes contacting the wet sensory section against the user and producing a sensory signal to the user.

According to another aspect of one or more embodiments of the present invention, a method of potty training a user, the method including providing a potty training liner in an unworn condition. In the unworn condition the potty training liner includes a central void region and a broad central channel. The method further includes applying the potty training liner to the user. In the worn condition, the potty training liner includes a closed central void region and a secondary void region having a pooling area for fluids released by the user.

Other aspects of the present invention will be apparent from the following description and claims.

### BRIEF DESCRIPTION OF THE DRAWINGS

FIG. 1 shows a top view of a potty training liner embodiment adapted for use by a girl.

FIG. 2 shows a transverse partial cross-sectional view taken near the rear liner wall of the FIG. 1 embodiment.

FIG. 3 shows a top view of a potty training liner embodiment adapted for use by a boy.

FIG. 4 shows a top view of a potty training liner embodiment adapted for use overnight.

FIG. 5 shows a transverse cross-sectional view taken through an embodiment.

FIG. 6 shows a top, side, and two cross-sectional views of another potty training liner embodiment.

FIG. 7 shows the FIG. 6 embodiment in perspective view.

FIG. 8 shows additional top, side, and perspective views of the FIG. 6 embodiments, flexed into an approximate “as-worn” configuration.

FIG. 9 shows a top, side, and two cross-sectional views of yet another potty training liner embodiment.

FIG. 10 is a cross-sectional view of a potty training liner according to embodiments of the present disclosure.

FIG. 11 is a cross-sectional view of a potty training liner according to embodiments of the present disclosure.

FIG. 12 is a top view of a potty training liner according to embodiments of the present disclosure.

FIG. 13 is a top view of a potty training liner according to embodiments of the present disclosure.

FIG. 14 is a top view of a potty training liner according to embodiments of the present disclosure.

FIG. 15 is a top view of a potty training liner according to embodiments of the present disclosure.

FIG. 16 is a top view of a potty training liner according to embodiments of the present disclosure.

FIG. 17 is a side cross-sectional view of a potty training liner according to embodiments of the present disclosure.

FIG. 18 is a side cross-sectional view of a potty training liner according to embodiments of the present disclosure.

FIG. 19 is a side cross-sectional view of a potty training liner according to embodiments of the present disclosure.

### DETAILED DESCRIPTION OF THE INVENTION

One or more embodiments of the present invention will now be described in detail with reference to the accompanying figures. For consistency, like elements in the various figures are denoted by like reference numerals. In the following detailed description of the present invention, specific details are set forth in order to provide a thorough understanding of the present invention. In other instances, well-known features to one of ordinary skill in the art are not described to avoid obscuring the description of the present invention.

Although pull-on type diapers do not keep children, who can sense that they need to go to the bathroom, from going, it is now believed that pull-on type diapers have characteristics that do not assist the child in progression from the security of a diaper to wearing unprotected underwear. Pull-on type diapers have a bulky “diaper” feel to them, like the traditional diapers a child has worn since birth. Accordingly, the use of pull-on type diapers for potty training can send confusing messages to children.

The present embodiments are designed for use with underwear, as a transition between traditional or pull-on type diapers and underwear alone. These embodiments are referred to herein as “potty training liners,” and consist of disposable inserts for use with underwear. Potty training liners are preferably designed to be used after a child gains some bladder control and is ready to progress to wearing real underwear. Underwear outfitted with potty training liners does not have the “diaper-feel” of pull-on diapers, and yet gives some protection against unimpeded urine flow onto clothing, carpets, and furniture, etc.

Potty training liners enable a child to wear real underwear earlier while reducing the risk of unnecessary embarrassment and clean-up. Most children do not want to soil their clothes and are often embarrassed at their mistake. A child who has some bladder control knows he “has to go,” but simply waits too long, or gets distracted and forgets he has to go. This typically results in a child not quite making it to the bathroom in time. Potty training liners can save a child in this situation from soiling their clothes, and other items, and the feel of the liner in use and after creates awareness prompting the child to take action earlier and allowing them to learn.

Potty training liners are designed for preferable use as “short-term” emergency protection, whereas diapers and sanitary pads are designed for “long-term” wearable protection. At least some preferred embodiments of potty training liners are not designed to keep wetness away from the child, or to allow a child to continue playing after having an accident, but simply keep wetness confined to the pad until they can get to a bathroom or get assistance.

It is now believed that the use of potty training liners will speed the process of potty training by getting children into real underwear much faster, and creating greater awareness of the child's need to go, when they first realize they should, without the added negative consequences of having an unprotected accident. Many busy parents keep children in diapers or pull-on diapers longer, because they fear the clean-up and hassle. Often, parents do not have the time to watch their child as closely as is required when transitioning to real underwear, and frequently forget to remind their child to go to the bathroom. Potty training liners makes it more convenient for parents to begin training earlier, helps the child “self-train,” and keeps the experience positive. Sanitary and incontinence pads currently exist in the marketplace for use by adults, but are not believed to meet the needs of a child learning bladder control. Perhaps most important, neither sanitary nor incontinence adult pads will fit or function properly for a child. Sanitary pads are developed to absorb slower, thicker body fluids and hold them over long periods of time. Likewise, incontinence pads are designed for incontinent adults, who typically produce small urinary leaks over time. Children in the potty training process typically have the bladder control to hold their urine until their bladder is nearly full, followed by an urgent release that produces a high volume of urine in a brief time period. Most importantly, adult incontinence pads are not designed to aid the potty training process because they do not have a dry lightweight feet required for training and differentiating the bulky diaper-like feel between a child's legs. Further, adult incontinence pads are designed to keep the user dry and comfortable and would not advance the potty training process.

The preferred embodiments do not protect the child from wetness, as this is not conducive to potty training, nor do they provide long-term wet wearability, like a diaper or other pad. The preferred embodiments do, however, contain several features designed to enhance the comfort and wearability of the dry liner. In preferred embodiments the size and shape, along with compressible side walls, give a comfortable, flexible fit for a small child while providing substantial protection from unexpected puddles on floors and furniture from a single insult. The flexible bottom and absorbent side wall as well as the alternative then flexible core design can retain large volumes of urine quickly, by containing and channeling the fluid, while also allowing fluid to stay in contact with the skin longer before final absorption into the core In addition, the core is designed to saturate earlier than existing products. The liners in some embodiments have a form fitting rise at the middle, and rear, of the liner in attempt to protect overflow along the natural curves of the body, while urine is absorbed. The liners can also have adhesive limited to the outer portions of the underside of the liner, so that the center can flex and provide expansion as urine is collected and absorbed. This design works with cloth undergarments which naturally give a little in the center.

Pull-on diapers disperse urine in the pant, providing long term wear-ability. This has the effect of numbing awareness and encouraging a child to continue playing and not address the situation. Children must learn to take action to prevent accidents. Part of this learning process is knowing “when” to act, especially when attention is focused on something exciting, like playing. The preferred liners collect urine more centrally, increasing awareness. As the preferred liner becomes saturated, it feels awkward and heavy between the child's legs, prompting the child to take action. Furthermore, because the dry liner is thin and comfortable in the child's underwear, the child distinguishes the liner as different than a diaper. However, once saturated, preferred embodiments swell considerably in size. The change, or increase in size, from dry and thin to wet and bulky helps signal the user to take action both during and after an accident.

Most absorbent products on the market are designed with at least three layers consisting of a liquid permeable top layer, an absorbent core, and a non-permeable bottom layer. Absorbent materials used in various absorbent products are known. Typically, such products comprise an absorbent fibrous matrix of cotton or wood pulp fluff enhanced with a high-absorbency material known as “superabsorbent.” Superabsorbents are typically crosslinked polymers capable of absorbing 10-100 times their weight in water. Superabsorbents are frequently used in diapers and personal care products to enhance absorption. There are many ways, known to those in the art, to combine superabsorbent and absorbent materials. For example, U.S. Pat. No. 5,356,403, issued to Faulks, et al., describes a layered fibrous structure laced with varying amounts of superabsorbent material at various densities. Other methods involve weaving the superabsorbent into a single fibrous pad during production, or sandwiching a superabsorbent between two fibrous layers.

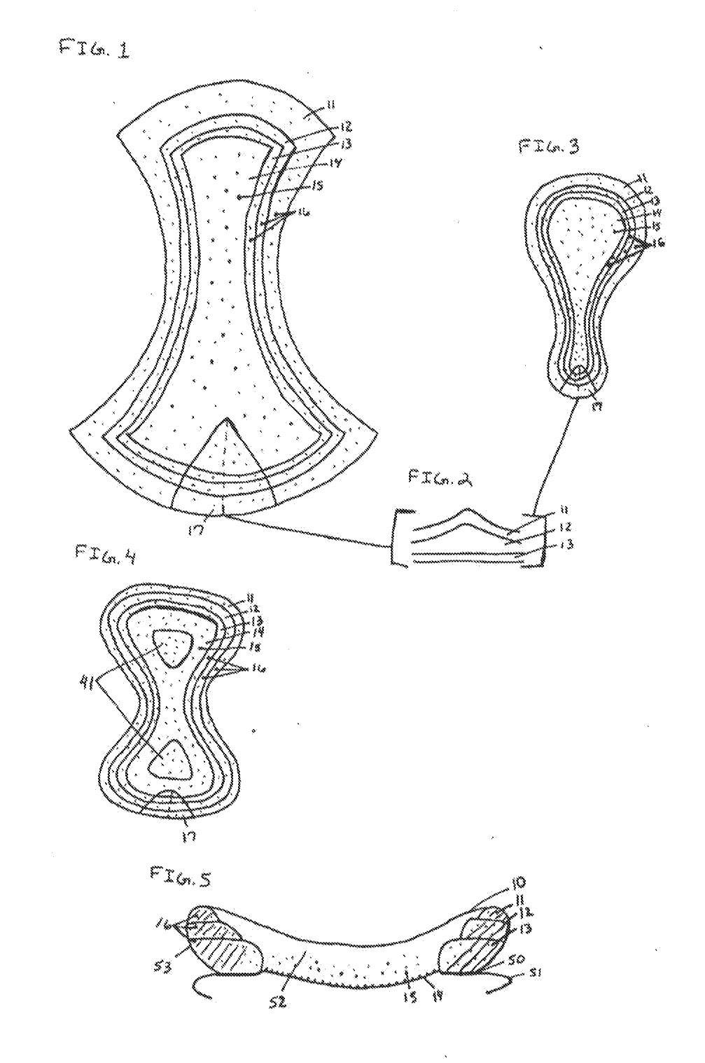

The preferred embodiments are illustrated in FIG. 1 (Top view of liner adapted for girls); FIG. 2 (partial rear transverse cross section); FIG. 3 (Top view of liner adapted for boys); FIG. 4 (Top view of liner adapted for use overnight); and, FIG. 5 (traverse cross-section). The first layer 10, (FIG. 5) comprises a thin, soft, absorbent top sheet, such as absorbent Rayon-polyester with low density, or other non-woven material, which covers and protects the underlayers (the top layer is removed in the drawings FIGS. 1, 2, 3, and 4). Urine moves immediately through this top layer with minimal resistance.

The bottom layer 14 is a soft flexible waterproof material which contains loose superabsorbent material, with or without sparse fibrous material mixed in to aid in preventing gel block. 15. The superabsorbent is typically a hydrophilic polymer made of fine particles of an acrylic acid derivative, such as sodium acrylate, potassium acrylate, or an alkyl acrylate. Further, the flexible bottom layer 14 has a light coating of adhesive on the inside to hold some of the powdered superabsorbent in place and to ensure coverage. The loose superabsorbent 15 allows gravity to dictate where it is needed most. The bottom 14 is designed to flex, this allows for inward compression of the sides for comfort and expansion and increased urine retention. Unlike current absorbent pads, cloth underwear gives and will allow additional expansion in the event of an accident.

The center of the liner 52 is substantially hollow, or concave, so that urine may flow easily into the space while allowing the top layer 10, and bottom layer 14, to sag when the sides are compressed creating space for urine to pool as it is being absorbed. Currently, when sanitary napkins or diapers are compressed on the side, the pad center has a tendency to bunch “up.” Without a fluid channel, this can create overflow, when fluid hits the surface faster than it can be absorbed or alternatively, as in most absorbent articles, it wicks fluid away immediately defeating the purpose of training to allow the wearer to feel the fluid and wetness along the skin. Allowing the center to compress downward, and the sides to compress inward by the thighs, provides a more comfortable fit, and affords space for urine to travel a short distance along the skin before being retained, while avoiding the stiff feel of a diaper or pad. A thin and flexible acquisition layer may be used to slow the movement of fluid if necessary. Further the top layer may be adhered to the bottom layers.

The center of the liner 52 is surrounded by a soft absorbent wall (11, 12, 13). The preferred wall configuration contains stacked absorbent and superabsorbent material in a stair-step fashion extending outward. The wall is made of three layers, a bottom layer 13, a middle layer 12 set off-center toward the outside, and a top layer 11 set off-center toward the outside. This creates a lower profile, downwardly and inwardly collapsible wall, that minimizes blockage of urine flow to the center of the liner when compressed between the thighs of the wearer in use. Each layer contains superabsorbent 16 which causes the wall to swell, expanding laterally and horizontally to further trap urine and also create movement that can be felt and acts as a signal to the trainer to take action. This configuration allows for sides to compress comfortably while absorbing and channeling fast streaming urine toward the center-front, center-rear and bottom-center of the liner where it can be contained until the superabsorbent has had time to work. Extra absorbent material in the liner mid-section wall 11, 12, 13 can provide additional protection along the natural curve of the body.

The fibrous absorbent material used in the wall (11, 12, 13) may be formed from natural or synthetic fibers and by using methods such as air laying, spunbond, meltblown, or any of the methods known to those skilled in the art for making absorbent fibrous materials. The fibrous layer contains a superabsorbent 16. The superabsorbent may be dispersed through the fibrous layer at the time the fibrous layer is created, or sandwiched between fibrous layers, or by any means available to those skilled in the art.

Additionally, the top two layers in the wall of the rear area 17 may have extra absorbent material, creating a form-fitting rise to help catch potential overflow to the rear when sitting or lying down.

Liners may be designed as unisex, but for comfort, and a lower profile, the preferred liners will be designed based upon gender specific needs. The boy's liner, FIG. 3, can be in a somewhat relaxed “sport-cup” shape, with a larger catch area in the front, and an extended reserve in the rear area.

Liners for Girls, FIG. 1, can be a more oblong hourglass shape, with a larger catch area in the rear, and a larger reserve area in the front, but will not extend as far in the front.

Liners designed for overnight protection, FIG. 4, will be longer, as well as contain extra absorbent material in the walls (11, 12, 13), and in the front and rear center 41 sections, to help catch urine while a child is lying down.

Liners designed for advanced trainers, who are almost finished with potty-training, will have thinner walls and a lower profile, to catch the little leaks that escape when they are trying to get to the bathroom.

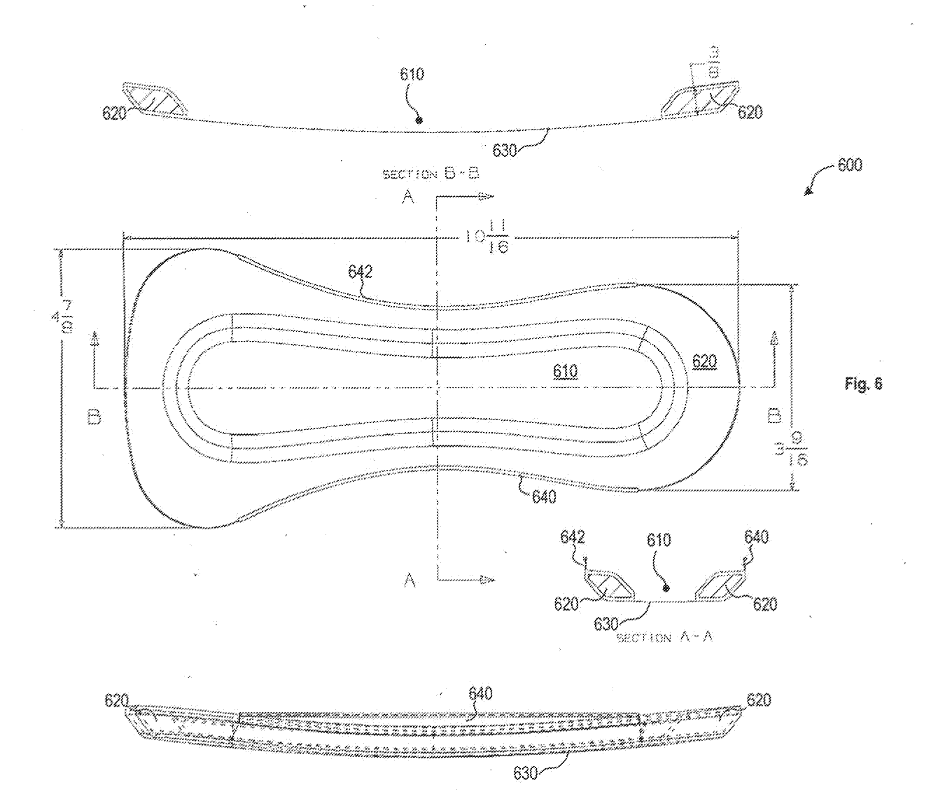

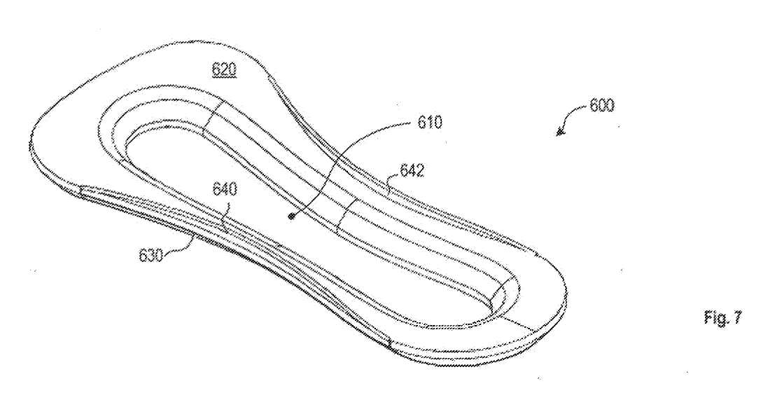

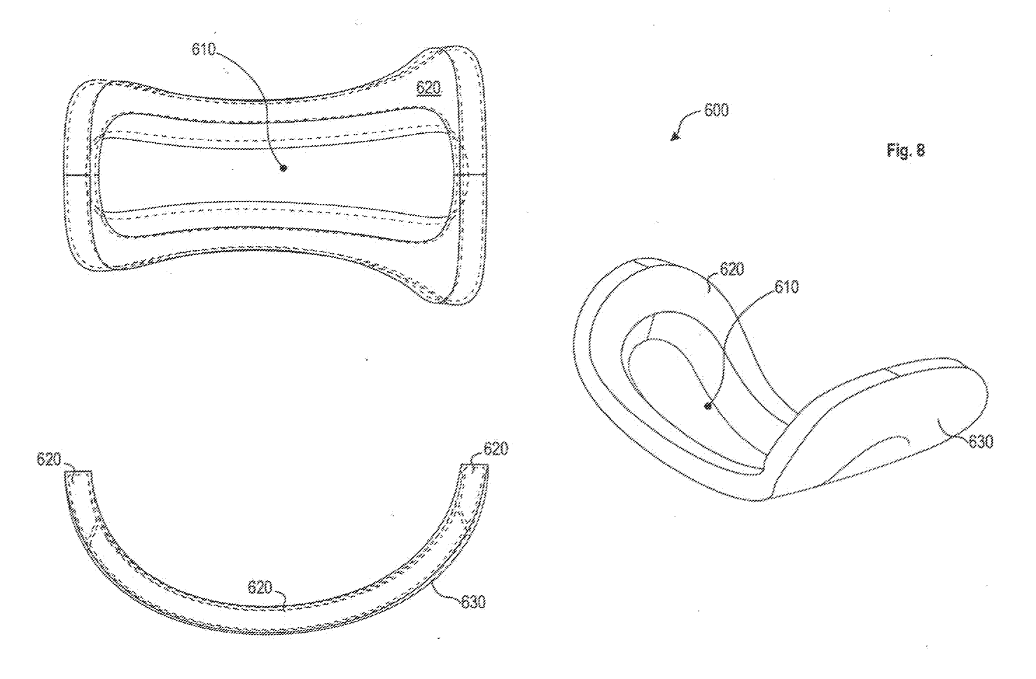

Colors can vary, and may include fun child-like designs, and/or a disappearing design, or positive reinforcement symbol on the inside top layer of the pad, as a positive indication that the pad is dry. An alternative to a pad incorporating absorbent layers is a formed pad 600 such as shown in FIGS. 6, 7, and 8. Formed pad 600 is machine-shaped and pressed into a liner 630 to leave a central void 610 with the bulk of the absorbent material contained in a peripheral region 620. The peripheral region 620 can be filled with superabsorbent, pulp, fluff, and combinations thereof. In this embodiment, the cross-section of the peripheral sidewalls has an approximately parallelogram shape to aid comfort. A water-permeable top sheet placed over the top surfaces of the pad 600 and pressed to them, e.g., with adhesive, can help the pad hold its shape. An outer side edge 640, 642 forms side gathers to help prevent leakage. The side gathers can be elasticized to help form a deployed pad to the child's shape, as shown in FIG. 8. Adhesive means (not shown) are applied to the underside of the liner or on side wings to adhere the pad to a child's undergarment. Preferably, a central region of the pad does not contain adhesive, allowing the pad to move naturally and fill space left by the undergarment's flexing.

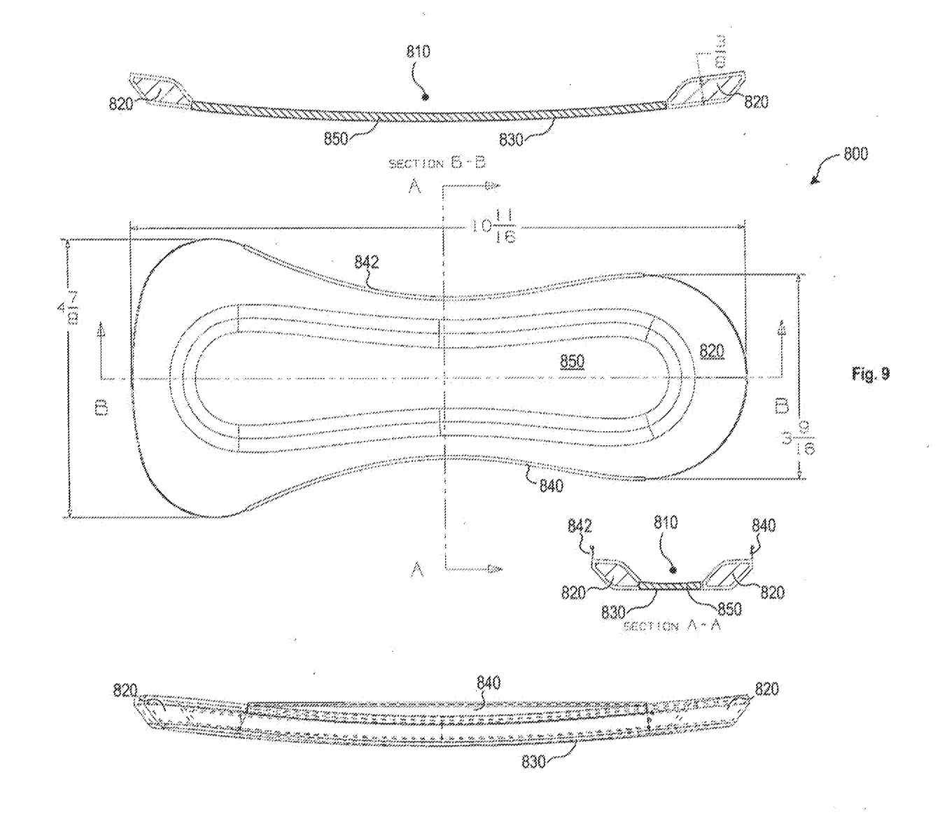

FIG. 9 shows an additional embodiment 800. Like embodiment 600, pad 800 has a central void 810, an absorbent peripheral region 820, a waterproof liner 830, and side gathers 840, 842. Instead of the top sheet being bonded directly to the waterproof liner in the central void region, however, pad 800 incorporated additional absorbent/superabsorbent material in a central absorption region 850, which underlies central void 810. Central absorption region can contain the same materials as the peripheral absorption region, or can use strictly superabsorbent or acquisition. Preferably, material in or over the central absorption region is relatively thin if employed, so that the center of the pad remains easily deformable and contains a large void region. The central absorption region 850 assists the peripheral absorption region 820 in absorbing fluid that is quickly pooled into the central void. Preferably, however, most of the absorbing capacity (e.g., 75% or more) remains in the peripheral region. The central void need not be completely bereft of material in a given embodiment. It can be filled, completely or partially, with an low-density material that has relatively little permanent fluid-holding capacity, as compared to the absorption regions of the pad, but low impedance to fluid flow when saturated. Such a low-density material could help the pad maintain its shape or slow lateral fluid movement and provide additional wet, unpleasant feedback to a child having an accident.

The absorbent sections of the liner can be formed, e.g., using known methods and materials from the art. For instance, pulp/fluff material formed in a drum can be pressed to the desired shape of the absorbent portions of the liner. A superabsorbent material or combination of superabsorbent materials can be used instead, and/or the superabsorbent material can be mixed with pulp/fluff or other absorbent and nonabsorbent material combinations. One example of a superabsorbent material is Infinicel™, made by Proctor and Gamble, Inc. An alternative is the absorbent material marketed as Nanosan®.

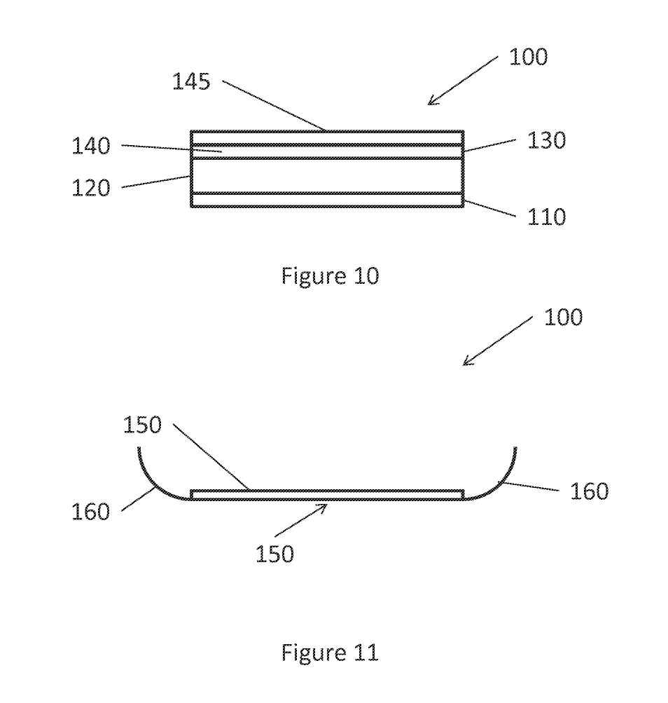

Referring to FIG. 10, a cross-sectional view of a potty training liner according to embodiments of the present disclosure is shown. In this embodiment, potty training liner 100 includes a hydrophobic bottom layer 110, a core 120 disposed on and above the hydrophobic bottom layer 110, and a top layer 130 having an acquisition layer 140, disposed on and above core 120.

Hydrophobic bottom layer 110 may be formed from various hydrophobic materials including various plastics, laminates, and the like. As used herein, hydrophobic includes material that tends to resist being wetted by water or other fluids. Different materials may have different hydrophobic tendencies. For example, some hydrophobic materials may repel water and thus may be capable of forming a substantially waterproof barrier. Other hydrophobic materials may be substantially water-resistant, but may over time absorb a certain percentage of water or other fluids. Still other hydrophobic materials may allow a certain percent of water through the material as a result of other aspects of the material such as, for example, the porosity of the material. Hydrophobic bottom layer 110 is configured to be substantially waterproof, thereby restricting the flow of water and water containing fluids therethrough.

Potty training liner 100 also includes core 120. As discussed above, core 120 may be formed from any absorbent material, most commonly a fibrous matrix of cotton or wood pulp fluff which may be enhanced with a high-absorbency material known as “superabsorbent.” Superabsorbents are typically crosslinked polymers capable of absorbing 10 to 100 times their weight in water. Superabsorbents may also be formed from various laminate structures. For example, a laminate superabsorbent material may include a layer of discrete discontinuous elements attached to a continuous porous support layer. The superabsorbent may be formed by saturating a porous substrate with an acrylic, such as, for example, an acrylic acid monomer solution. The monomer may then be polymerized and crosslinked in the monomer web, thereby allowing the resultant superabsorbent material to absorb a desired fluid. In the present disclosure, the superabsorbent is designed to absorb a water-based fluid, such as urine.

In certain embodiments, core 120 may be selected to have minimal absorptive properties. As potty training liner 100 is configured to promote awareness in a user, by decreasing the absorptive properties of the core 120, a greater volume of fluids may be retained in the portions of potty training liner 100 above core 120, i.e., acquisition layer 140 and top layer 130. For example, rather than allow the majority of fluid to be absorbed by core 120, core absorption could be limited to 50-80 milliliters, 80-125 milliliters, 125-160 milliliters, or less than 200 milliliters. Additionally, core 120 may be selected to decrease the absorbency rate. For example, core 120 that absorbs less fluid over time allows more fluid to stay in contact with the user, while still absorbing the fluid over an acceptable time period. Examples of acceptable absorption rates may include rates less than 5 ml/min, 5-10 mL/min, 10-20 mL/min, or greater than 20 ml/min, depending on how long the sensation of wetness is desired to affect the user.

Potty training liner 100 also includes top layer 130. Top layer 130 is disposed above core 120 and may by hydrophilic. As used herein, hydrophilic refers to a material that has an affinity for water or water-based fluids. Top layer 130 may also be substantially porous, thereby allowing water or water-based fluids, such as urine, to flow through top layer 130 to core 120. In certain embodiments, top layer 130 may by hydrophobic with a metered degree of porosity. For example, top layer 130 may be formed from a substantially hydrophobic material, thereby causing fluids to be retained on top layer 130. However, top layer may also include a number and size of pores, thereby allowing fluids to flow through at a desired rate. Those of ordinary skill in the art will appreciate that the rate at which fluids pass through top layer 130 may be selected in order to achieve optimal potty training, which is discussed in greater detail below.

Top layer 130 also includes acquisition layer 140. Acquisition layer 140 may be hydrophilic in order to absorb a certain percentage of the fluid or may be hydrophobic with a high porosity in order to allow the fluid to pass through acquisition layer 140 to core 120. In certain embodiments, acquisition layer 140 may be a non-woven material with enhanced capillary action, thereby allowing fluids to distribute evenly thereon. In certain embodiments, acquisition layer 140 may include materials that are optimized to retain a “wet feel,” thereby enhancing the training benefits of potty training liner 100, which is discussed in greater detail below.

Additionally, acquisition layer 140 may be configured to distribute the fluid to certain sections of potty training liner 100. For example, in certain embodiments, acquisition layer 140 may be configured to distribute the fluid in a substantially equal portion to all sections of potty training liner 100. In other embodiments, acquisition layer 140 may be configured to concentrate the fluid into specific sections of potty training liner. Depending on the configuration of acquisition layer 140, the absorption rate of core 120 may vary. For example, acquisition layer 140 concentrates the fluid to a respectively small area, fluid absorption by core 120 may be slowed, thereby retaining fluid above core 120 of potty training liner 100 for a longer period of time. In other embodiments, acquisition layer 140 may distribute fluids over a larger area, thereby increasing the absorption rate of core 120. Those of ordinary skill in the art will appreciate that in certain embodiments, the configuration of top layer 130, bottom layer 110, core 120, and acquisition layer 140 may vary depending on the requirements for potty training liner 100. For example, in one embodiment, no acquisition layer 140 may be required. In such an embodiment, top layer 130 may provide the functionality of acquisition layer 140. Similarly, in other embodiments, core 120 may provide the functionality of acquisition layer 140. In still other embodiments, core 120 and acquisition layer 140 may be included as a single layer that is capable of absorbing and distributing fluids. Additionally, in certain embodiments, bottom layer 110 may be a material applied to core 120, acquisition layer 140, etc. In such an embodiment, a waterproof layer may be applied to one side of core 120, thereby preventing fluids from flowing out of core 120.

Potty training liners 100 also include a hydrophilic sensory section, which in this embodiment is a hydrophilic sensory strip 145 disposed on top layer 130. As illustrated, hydrophilic sensory strip 145 is shown as being disposed across all of top layer 130, however, those of ordinary skill in the art will appreciate that in certain embodiments, hydrophilic sensory strip 145 may be disposed on a small area of top layer 130. For example, in certain embodiments, hydrophilic sensory strip 145 may be disposed on between 5 and 10 percent of the total area of top layer 130. In other embodiments, hydrophilic sensory strip 145 may be disposed on between 10 and 25 percent of the total area of top layer 130. In other embodiments, hydrophilic sensory strip 145 may be disposed on less than 10 percent of the total area of top layer 130. In still other embodiments, hydrophilic sensory strip 145 may be disposed on greater than 25 percent of the total area of top layer 130. One of ordinary skill in the art will appreciate that a hydrophilic sensory strips 145 disposed to cover any area of top layer 130 are within the scope of the present disclosure.

Additionally, the geometry and location of hydrophilic sensory strip 145 may be selected in order to impart a certain sensation to the user. For example, hydrophilic sensory strip 145 could be elongated and narrow in order to impart a sensation over a relatively narrow latitudinal area. Alternatively, hydrophilic sensory strip 145 could be latitudinally expanded in order to cover a wider area of the user.

Hydrophilic sensory strip 145 is optimized to absorb and retain a portion of the fluid. Examples of materials that may be used to form hydrophilic sensory strip 145 include cotton, naturally occurring hydrophilic fibers, synthetically manufactured hydrophilic fibers, or other shapeable hydrophilic materials. During use, hydrophilic sensory strip 145 is configured to absorb and retain a portion of the fluid it contacts. Thus, as fluid flows over top layer 130 and into acquisition layer 140, a certain portion of the fluid remains above core 120 prior to absorption. As the fluid is retained above core 120 prior to absorption, hydrophilic sensory strip 145 becomes damped or wetted. Because top layer 130 may cause fluids to pass slowly therethrough, hydrophilic sensory strip 145 may be in contact with fluids for a prolonged period of time, thereby keeping hydrophilic sensory strip 145 wet. Additionally, hydrophilic sensory strip 145 may be configured to retain fluid for a period of time longer than the time fluid is present in acquisition layer 140, or top layer 130. Thus, even after substantially all of the fluid has passed through top layer 130, acquisition layer 140, and into core 120, hydrophilic sensory strip 145 remains wet.

Depending on the requirements of the hydrophilic sensory strip 145, hydrophilic sensory strip 145 may be configured to stay wet for a desired amount of time. For example, in certain embodiments, hydrophilic sensory strip 145 may be configured to stay wet for approximately 15 minutes. In other embodiments, hydrophilic sensory strip 145 may be configured to stay wet for between 5 and 15 minutes. In other embodiments, hydrophilic sensory strip 145 may be configured to stay wet for between 15 and 30 minutes. In still other embodiments, hydrophilic sensory strip 145 may be configured to stay wet for greater than 30 minutes. Those of ordinary skill in the art will appreciate that the time period in which the hydrophilic sensory strip 145 is configured to stay wet may vary depending on the stage of training of the user, the user's age, the user's health condition, etc.

In certain embodiments, hydrophilic sensory strip 145 may be connected to top layer 130 though use of a waterproof adhesive (not independently illustrated). Use of a water proof adhesive below, or at the base of hydrophilic sensory strip 145 may decrease the absorption of fluids out of hydrophilic sensory strip 145. Said another way, because the waterproof adhesive is in direct contact with top layer 130 and hydrophilic strip 145 is not directly contacting top layer 130, top layer 130 does not wick moisture out of hydrophilic strip 145 as quickly. Because moisture is not wicked out of hydrophilic strip 145 as quickly, hydrophilic strip 145 may stay wet for a longer period of time, thereby enhancing the training properties of potty training liner 100.

In certain embodiments, potty training liner 100 may include a sensory section (not independently illustrated). For example, the sensory section could be a hydrophilic sensory strip 145, a portion of top layer 130, a portion of acquisition layer 140, a portion of core 120, and/or a portion of bottom layer 110. To further illustrate such an embodiment, in one embodiment, sensory section may include a defined area of top layer 130, that while not an independent component of potty training liner 100, includes a texture, material, etc., that provides a sensory signal to a user. Examples of sensory sections may include a section of potty training liner 100 that provides a tactile signal or sensation to a user to let the user know that an incident has occurred.

A method of manufacturing a potty training liner 100 according to embodiments of the present disclosure may include the following steps. Bottom layer 110 may be formed of an appropriate geometry to match the requirements of a user. Core 120 may be attached to bottom layer 110. In certain embodiments, core 120 may be attached to bottom layer 110 through adhesive, such as waterproof adhesives. In other embodiments, core 120 may not be attached to bottom layer 100, but instead may be placed over but not directly connected to bottom layer 110.

Top layer 130 may then be disposed on top of core 120. Top layer 130 may be connected to core 120 though adhesives, or otherwise may be connected to bottom layer 110, with core 120 being disposed therebetween. Top layer 130 may be connected to bottom layer 110 directly using adhesive, or otherwise mechanically connected through the use of, for example stitching. Acquisition layer 140 may be a portion of top layer 130 or be disposed between core 120 and top layer 130. In such an embodiment, acquisition layer 140 may be connected to either top layer 130 and/or core 120 though adhesives, or otherwise may be placed between top layer 130 and core 120 and be held in place by connecting top layer 130 to core 120, as described above. In other embodiments, acquisition layer 140 may be an integral part of top layer 130 and the connection of top layer 140 to either core 120 or bottom layer 110 may connect acquisition layer 140.

Hydrophilic sensory strip 145 may be connected to top layer 130 through the use of waterproof adhesives, as described above. In other embodiments, hydrophilic sensory strip 145 may be connected to top layer 130 through mechanical means, such as stitching, or otherwise be connected through other methods for connecting two materials that are known in the art. In still other embodiments, hydrophilic sensory strip 145 may be an integral part of top layer 130, thereby not requiring independent connection thereto.

Referring to FIG. 11, a cross-sectional view of a potty training liner 100 according to embodiments of the present disclosure is shown. In this embodiment, potty training liner 100 is shown with a central region 150 and standing side-gathers 160. Central region 150 includes bottom layer (110 of FIG. 10), core (120 of FIG. 10), and top layer (130 of FIG. 10), the individual components are not independently illustrated for clarity in the figure. As illustrated, central region 150 may have a substantially linear, or flat, cross-section prior to being worn by a user. In other embodiments, central region 150 may have an angled or arcuate cross-section in order to enhance the fit of the potty training liner on the user.

In still other embodiments, the sides of central region 150 may be stepped in order to direct fluid flow in a predetermined direction. In such an embodiment, the sidewalk of central region may include one or more steps, thereby directing fluid to the center of the potty training liner 100. In an embodiment having a substantially flat cross-section, when worn, the center region 150 may form into an arcuate shape, and depending on the selected geometry, fluid may be distributed more evenly within central region 150 or may be directed to a predetermined location on central region. In still other embodiments, central region 150 may have a sloped or arcuate cross-section prior to being worn by the user. Such a sloped or arcuate cross-section may be configured to enhance the wearability and/or comfort of the user.

Generally, the geometry of central region 150 may vary depending on the desired flow dynamics of potty training liner 100. Those of ordinary skill in the art will appreciate that potty training liners 100 configured to have a pooling effect toward the center 170 of center region 150 may have stepped or higher angled sides, either before or during wear. Additionally, potty training liners 100 having a more flat central region 150 may be optimized to distribute fluids more evenly or, depending on the materials used, to distribute fluids to a particular location.

Potty training liner 100 also includes standing side-gathers 160, which are connected to one or more of the bottom layer (110 of FIG. 10), core (120 of FIG. 10), top layer (130 of FIG. 10) and/or acquisition layer (140 of FIG. 10). Standing side-gathers 160 are configured to pull the sides of one or more of the bottom sheet, core, top layer and/or acquisition layer into contact with the skin of the user and prevent fluid overflow. During use, standing side-gathers 160, pull the sides of potty training liner 100 upwardly, thereby causing core to flex to conform to the profile of the user. Because the potty training liner 100 bends to fit the profile of the user, standing side gathers 160 are in contact with the users skin as well as at least one of the bottom layer (110), core (120), top layer (130) and/or acquisition layer (140) against the user, thereby preventing fluids from leaking out of the potty training liner 100. For example, in certain embodiments, during use, potty training liner 100 bends into a general U-shape, matching the profile of the user. When fluid is released into the potty training liner 100, the standing side-gathers 160 prevent overflow of the fluid out of potty training liner 100.

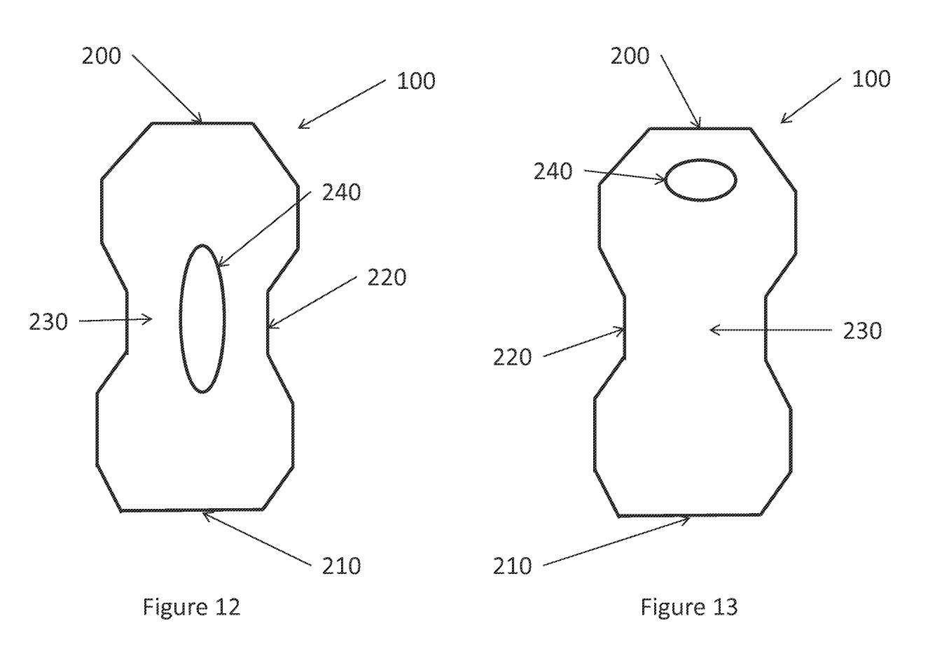

Referring to FIG. 12, a top view of a potty training liner 100 according to embodiments of the present disclosure is shown. In this embodiment, potty training liner 100 is shown having a front portion 200, a back portion 210, a side portion 220, and a central region 230. Those of ordinary skill in the art will appreciate that the dimensions and geometry of potty training liner 100 may vary according to, for example, the age of the user, the weight of the user, the height of the user, the absorbency required, the types of materials used, etc. However, generally, potty training liner 100 includes expanded front and back portions 200/210, and a relatively narrower central region 230. Potty training liner 100 also includes a hydrophilic sensory strip 240.

As discussed above, potty training liner 100 may include a hydrophilic sensory strip 240 located at various locations on top layer (130 of FIG. 10). The location of hydrophilic strip 240 may vary depending on the requirements for the particular potty training liner 100. Examples of parameters that may affect the location of hydrophilic sensory strip 240 may include the sex of the user, the age of the user, the stage of potty training of the user, the height of the user, the weight of the user, etc. As illustrated in FIG. 12, hydrophilic sensory strip 240 may be located in the proximate center of central region 230. The geometry of hydrophilic sensory strip 240 may vary depending on the requirements of the user. For example, in certain embodiments, hydrophilic sensory strip may be relative narrow and elongated, however, in other embodiments hydrophilic sensory strip may be relatively wide but short.

Referring to FIG. 13, a top view of a potty training liner 100 according to embodiments of the present disclosure is shown. In this embodiment, potty training liner 100 is shown having a front portion 200, a back portion 210, a side portion 220, and a central region 230. Those of ordinary skill in the art will appreciate that the dimensions and geometry of potty training liner 100 may vary according to, for example, the age of the user, the weight of the user, the height of the user, the absorbency required, the types of materials used, etc. However, generally, potty training liner 100 includes expanded front and back portions 200/210, and a relatively narrower central region 230. Potty training liner 100 also includes a hydrophilic sensory strip 240.

As discussed above, potty training liner 100 may include a hydrophilic sensory strip 240 located at various locations on top layer (130 of FIG. 1). The location of hydrophilic strip 240 may vary depending on the requirements for the particular potty training liner 100. As illustrated in FIG. 13, hydrophilic sensory strip 240 may be located towards the front area of central region 230. The geometry of hydrophilic sensory strip 240 may vary depending on the requirements of the user. In this embodiment, hydrophilic strip 240 is located toward the front area, thereby causing the user to feel wetness in a relatively small area.

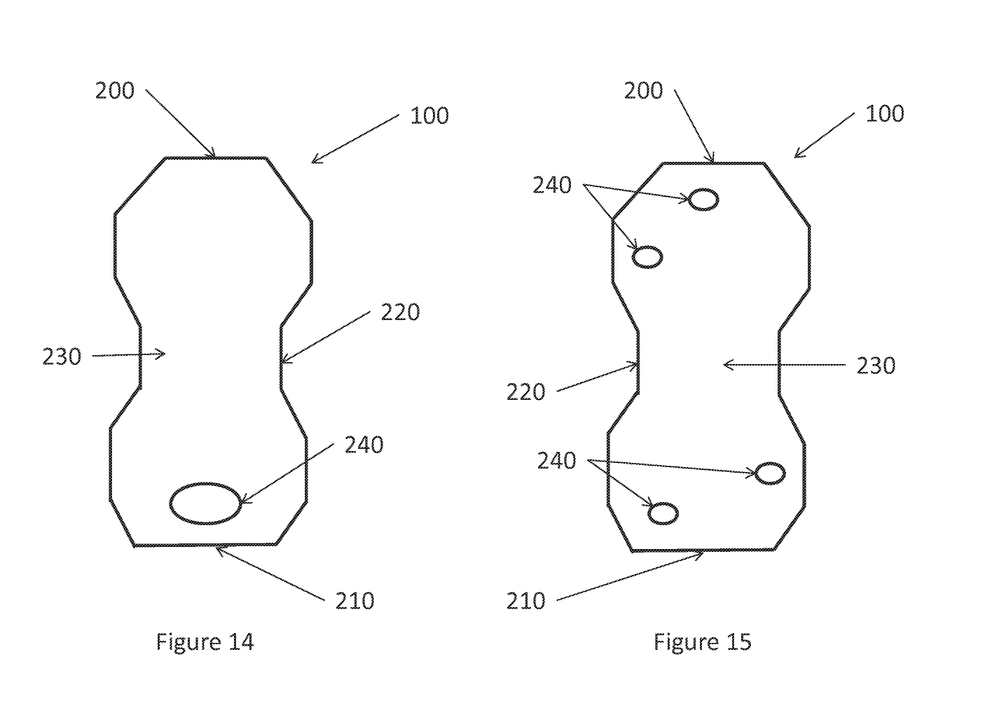

Referring to FIG. 14, a top view of a potty training liner 100 according to embodiments of the present disclosure is shown. In this embodiment, potty training liner 100 is shown having a front portion 200, a back portion 210, a side portion 220, and a central region 230. Those of ordinary skill in the art will appreciate that the dimensions and geometry of potty training liner 100 may vary according to, for example, the age of the user, the weight of the user, the height of the user, the absorbency required, the types of materials used, etc. However, generally, potty training liner 100 includes expanded front and back portions 200/210, and a relatively narrower central region 230. Potty training liner 100 also includes a hydrophilic sensory strip 240.

As discussed above, potty training liner 100 may include a hydrophilic sensory strip 240 located at various locations on top layer (130 of FIG. 1). The location of hydrophilic strip 240 may vary depending on the requirements for the particular potty training liner 100. As illustrated in FIG. 14, hydrophilic sensory strip 240 may be located toward the back of central region 230. The geometry of hydrophilic sensory strip 240 may vary depending on the requirements of the user, in this embodiment, hydrophilic strip 240 is located toward the front area, thereby causing the user to feel wetness in a relatively small area.

Referring to FIG. 15, a top view of a potty training liner 100 according to embodiments of the present disclosure is shown. In this embodiment, potty training liner 100 is shown having a front portion 200, a back portion 210, a side portion 220, and a central region 230. Those of ordinary skill in the art will appreciate that the dimensions and geometry of potty training liner 100 may vary according to, for example, the age of the user, the weight of the user, the height of the user, the absorbency required, the types of materials used, etc. However, generally, potty training liner 100 includes expanded front and back portions 200/210, and a relatively narrower central region 230. Potty training liner 100 also includes a hydrophilic sensory strip 240.

As discussed above, potty training liner 100 may include a hydrophilic sensory strip 240 located at various locations on top layer (130 of FIG. 10). The location of hydrophilic strip 240 may vary depending on the requirements for the particular potty training liner 100. As illustrated in FIG. 15, hydrophilic sensory strip 240 may be located throughout various sections of central region 230. In this embodiment, 4 discrete asymmetrical hydrophilic sensory strips 240 are disposed randomly within central region 230. In other embodiments, more or fewer hydrophilic sensory strips 240 may be used. For example, in certain embodiments, two, three, or more than 4 discrete hydrophilic sensory strips 240 may be disposed on central region 230. In still other embodiments, hydrophilic sensory strips 240 of different size may be used. In still other embodiments, hydrophilic sensory strips may cover substantially the entire central region 240. Those of ordinary skill in the art will appreciate the number, location, geometry, absorption rate, etc. may all vary according to embodiments of the present disclosure.

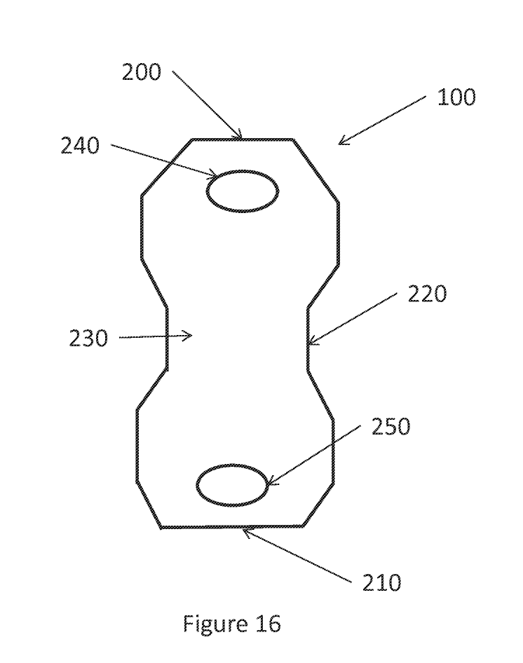

Referring to FIG. 16, a top view of a potty training liner 100 according to embodiments of the present disclosure is shown. In this embodiment, potty training liner 100 is shown having a front portion 200, a back portion 210, a side portion 220, and a central region 230. Those of ordinary skill in the art will appreciate that the dimensions and geometry of potty training liner 100 may vary according to, for example, the age of the user, the weight of the user, the height of the user, the absorbency required, the types of materials used, etc. However, generally, potty training liner 100 includes expanded front and back portions 200/210, and a relatively narrower central region 230. Potty training liner 100 also includes a hydrophilic sensory strip 240.

The hydrophilic sensory strip 240 used in this embodiments may include any variation of hydrophilic sensory strip 240 discussed above. As illustrated, hydrophilic sensory strip 240 in this embodiment is a relative small region toward the front of potty training liner 100. In addition to hydrophilic sensory strip 240, potty training liner 100 also includes a secondary sensory strip 250. Secondary sensory strip 250 is located toward back portion 210, however, those of ordinary skill in the art will appreciate that secondary sensory strip 250 may be located at various locations on central region 230, such as those described above with respect to hydrophilic sensory strip 250.

Secondary sensory strip 250 may be configured to provide additional types of sensory feedback to the user. For example, in one embodiment, secondary sensory strip 250 may be configured to provide a vibratory or auditory signal to the user. In such an embodiment, secondary sensory strip 250 may be configured to provide uncomfortable movement or produce an undesired sound. In one embodiment, secondary sensory strip 250 may comprise one or more sugars or salts that when contacted by fluids, such as urine, result in a vibratory response. In other embodiments, secondary sensory strip 250 may include materials that, upon contact with fluid, creates a popping or hissing noise. In addition to sugars and salts, other materials may include, for example, a gasified material and/or carbon dioxide and sorbitol.

Those of ordinary skill in the art will appreciate that the specific materials used to form secondary sensory strip 250 may vary in accordance with the type of response desired. In other embodiments, secondary sensory strip 250 may be provided to cause a tactile sensory response. Examples of secondary sensory strips 250 that may cause a tactile response may be materials that become rough to the touch upon contact with fluid, generate a temperature differential upon contact with fluid, or cause another sensory feel that is uncomfortable for the user.

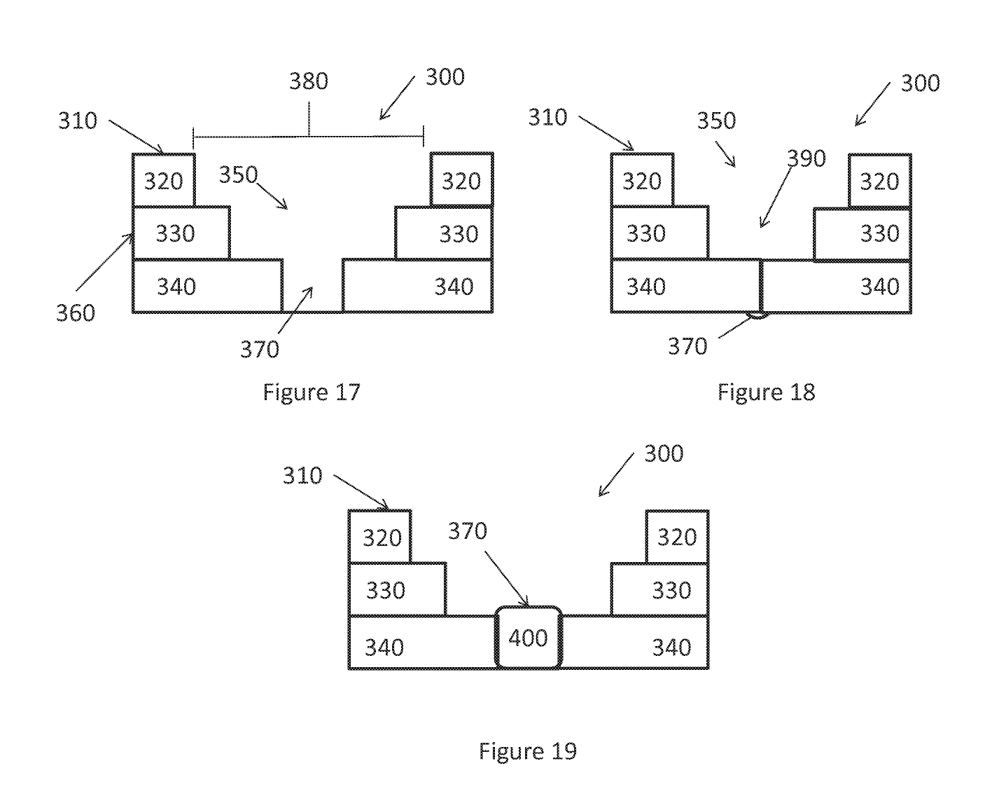

Referring to FIG. 17, a cross-sectional view of a potty training liner 300 in accordance with embodiments of the present disclosure is shown. In this embodiment, potty training liner 300 is shown prior to being worn by a user. Potty training liner 300 includes side walls 310. As shown, side walls 310 are segmented into three sections, a top layer 320, a middle layer 330, and a bottom layer 340. In other embodiments, side walls 310 may only include a single layer, may have two layers, or may have more than three layers. In this embodiment, the top layer 320, middle layer 330, and bottom layer 340 are disposed in a stair-stepped configuration. Said another way, bottom layer 340 extends toward a central region 350 more than middle layer 330 and top layer 320. Likewise, middle layer 330 extends toward central region 350 more than top layer 320. As illustrated, top layer 320, middle layer 330, and bottom layer 340 have a common outer side, thereby forming the external area 360 of side walls 310. In other embodiments, layers 320, 330, 340 may be offset externally, thereby forming an arcuate external area 360 of side walls 310. Similarly, rather than use a stair-stepped profile, layers 320, 330, 340 may be tapered, thereby forming an arcuate inner profile. Layers 320, 330, 340 may be formed from various materials, in certain embodiments, layers 320, 330, 340 may be configured to absorb a defined percentage of fluid, while in other embodiments, layers 320, 330, 340 may be substantially hydrophobic, thereby resisting fluid absorption.

In certain embodiments, the steps of potty training liner 300 may only occur in a particular region. For example, the steps may only occur in the center of potty training liner 300, while the front and back includes a substantially flat profile. In other embodiments, the center of potty training liner 300 may be stepped, while the front and back of the liner includes a substantially arcuate profile. Those of ordinary skill in the art will appreciate that in stair-stepped embodiments, the steps may be formed by stacking one or more layers of airlaid strips in the center of potty training liner 300. In certain embodiments, the steps may also be formed as a single molded sloped area.

Prior to being worn, potty training liner 300 has a central void region 370. The central void region 370 may include a superabsorbent material, such as those described above. For example, central void region 370 may define a core formed of superabsorbent material that has a relatively thin profile prior to wetting. The central void region 370 may thus be configured to swell and extend latitudinally and longitudinally when contacted by a fluid. Prior to being worn, potty training liner 300 may further include a relatively broad central channel 380. Those of ordinary skill in the art will appreciate that in an unworn state, potty training liner 300 may be relatively flexible and compressible. The flexibility and compressibility may change after contact with a fluid.

Referring to FIG. 18, a cross-sectional view of a potty training liner 300 in accordance with embodiments of the present disclosure is shown. In this embodiment, potty training liner 300 is shown while being worn by a user prior to contact with a fluid. Potty training liner 300 includes side walls 310. As shown, side walls 310 are segmented into three sections, a top layer 320, a middle layer 330, and a bottom layer 340. In other embodiments, side walls 310 may only include a single layer, may have two layers, or may have more than three layers.

When worn, sidewalls 310 compress inwardly, thereby blocking central void region 370. Said another way, bottom layer 340 may be configured such that both sides of bottom layer 340 contact in the central region 350, thereby substantially closing central void region 370. The contact between bottom layers 340 thereby forms a secondary void area 390. Because superabsorbent material of central void region 370 is thin and flexible when dry, central void region 370 may compress downwardly. In the worn and dry condition, potty training liner 300 remains relatively flexible and compressible, thereby not impeding action of the user.

The secondary void area 390 may provide a pooling area for fluids released by a user. Thus, upon release of a fluid, the fluid may temporarily pool in secondary void area 390 prior to absorption by one or more of the sidewalk 310 and/or the core of central void region 370. Over time, movement of the user may allow some of the fluid to flow through secondary void area 390 into the superabsorbent. Those of ordinary skill in the art will appreciate that the time fluid remains in secondary void area 390 may be controlled based on the materials used in forming sidewalk 310, the compressibility of sidewalls 310, the movement of the user, the absorption rate of the superabsorbent, etc. In certain embodiments, fluid may pool in secondary void area 390 for one to five minutes, five to ten minutes, or for greater than 10 minutes.

Referring to FIG. 19, a cross-sectional view of a potty training liner 300 in accordance with embodiments of the present disclosure is shown, in this embodiment, potty training liner 300 is shown while being worn by a user after the user has released a fluid into potty training liner 300. Potty training liner 300 includes side walls 310. As shown, side walls 310 are segmented into three sections, a top layer 320, a middle layer 330, and a bottom layer 340. In other embodiments, side walls 310 may only include a single layer, may have two layers, or may have more than three layers. Potty training liner 300 also includes a core 400, which is formed to include a superabsorbent.

After fluid contacts the core 400 for a defined time period, the core 400 may swell. The amount of time it takes for core 400 to swell may depend on the materials used, the amount of fluid released, the absorption rate of core 400, the absorption of sidewalls 310, etc. Those of ordinary skill in the art will appreciate that core 400 may swell to a defined size. For example, in certain embodiments, core 400 may be configured to swell 10-20%, 20-30%, 30-50%, 50-75%, 75-100%, or greater than 100%.

The swelling of core 400 thereby forces sidewalk 310 apart reopening central void region 370. Because central void region 370 is open, residual fluid may pool therein, thereby contacting the user. As explained above, the time fluid pools within central void region 370 may depend on the materials used, the amount of fluid released, the absorption rate of core 400, the absorption of sidewalk 310, etc. Additionally, the expansion of core 400 may contact the user, thereby provide a tactile feedback to the user that an incident has occurred.

In addition to providing a wetness and/or tactile feedback to the user, the expansion/swelling of core 400 forces sidewalk 310 apart. Because the sidewalk 310 do not compress inward as easily, they are bulkier and thus less comfortable for the user to wear. The bulk of potty training liner 300 may provide an additional signal to the user that an incident has occurred.

Those of ordinary skill in the art will appreciate that in certain embodiments, potty training liners 300 in accordance with the present disclosure may include other option features. For example, in certain embodiments, potty training liners 300 may also include an acquisition layer, side gathers, sensory strips, secondary sensory strips, etc., such as those discussed above with respect to other embodiments.

Embodiments of the present disclosure may be used in potty training users. Typically, the user will be a child going through potty training, however, in certain embodiments, users may be adults that for various reasons require later in life potty training.

Methods of potty training according to embodiments of the present disclosure include a user wearing a potty training liner, such as the liner discussed above. During an incident, a user may release a flow of fluid including a water-based fluid, such as urine. As the fluid is released into the potty training liner, the fluid contacts a hydrophilic sensory strip disposed in the potty training liner. Hydrophilic sensory strips may include strips discussed in the embodiments disclosed above. The hydrophilic sensory strip is thus contacted by the fluid, which wets the hydrophilic sensory strip.

The hydrophilic sensory strip may be moistened to a desired level of wetness. The wet hydrophilic sensory strip is then contacted against the body of the user. Depending on the location of the hydrophilic sensory strip within the potty training liners, the strip may contact the user in various locations. As discussed above, the strip may contact the user in a single location or in multiple locations. Additionally, in certain embodiments the hydrophilic sensory strip may be configured to swell upon contact with the fluid. The hydrophilic strip may then swell to a larger size causing the contact with the user. In certain embodiments, prior to contacting the hydrophilic sensory strip with the fluid, the hydrophilic sensory strip may not be in contact with the user. After wetting the hydrophilic sensory strip, the strip may swell to a size that contact the user. In certain embodiments, the hydrophilic sensory strip, after wetting, may come into occasional contact with the user, while in other embodiments, the wet hydrophilic sensory strip may swell to a size that substantially constantly contacts the user. In the later embodiment, wherein the hydrophilic sensory strip is in substantially constant with the user, the time the strip remains in contact with the user may vary. For example, in certain embodiments, the hydrophilic sensory strip may be configured to remain in contact with the user for a predetermined amount of time. For example, in certain embodiments, hydrophilic sensory strip may be configured to stay wet for approximately 15 minutes. In other embodiments, hydrophilic sensory strip may be configured to stay wet for between 5 and 15 minutes. In other embodiments, hydrophilic sensory strip may be configured to stay wet for between 15 and 30 minutes. In still other embodiments, hydrophilic sensory strip may be configured to stay wet for greater than 30 minutes.

The time the hydrophilic sensory strip remains in contact with the user may vary based on a condition of the user. For example, the age, stage in potty training, health condition, etc., of the user may require the hydrophilic strip stay in contact with the user for a greater or lesser amount of time. Additionally, in certain embodiments, the hydrophilic sensory strip may remain wet and only well partially. In such an embodiment, the hydrophilic sensory strip may only contact the user occasionally.

The contact between the wet hydrophilic sensory strip and user may thus produce a sensory signal to the user. A sensory signal, as defined herein, is a sensation provided to the user in order to elicit a response. In one embodiment, the sensory signal may include a wetness to inform the user that an incident has occurred. In other embodiments, the sensory signal may be other types to inform the user that an incident has occurred in order to elicit a response from the user.

In the instance where the sensory signal produced to the user is a wetness, the sensory signal may provide wet and uncomfortable contact to the user for a predetermined amount of time, as described in detail above. Other types of sensory signals may also be generated. For example, in one embodiment, a general tactile signal may be produced. Tactile signals may include, for example, a wetness, a loss of heat, a physical uncomfortable state, etc.

In embodiments where the sensory signal is a loss of heat, the user may experience a cold feeling that is different and/or uncomfortable. The loss of heat may occur in various ways. In one embodiment, the design of the potty training liners disclosed herein provides the loss of heat due to the construction of the liner. As explained above, the potty training liners are designed to be worn under undergarments as a replacement for traditional diapers. Undergarments used with potty training liners tend to be made from cotton or other natural fibers that allow the passage of air therethrough, unlike diapers and other undergarments that may restrict the flow of air. As the fluid in the potty training liners contacts the body of the user, and air flows around the potty training liners, heat is lost from the potty training liners and thus the body of the user. The loss of heat is perceived by the body as a cold sensation. Thus, when an incident occurs, the user may perceive an uncomfortable loss of heat, or coldness, as a sensory signal, sensation, thereby informing the user of the incident.

In another embodiment, the sensory signal may be a physical uncomfortable state. In addition to wetness providing a physical uncomfortable state, other forms of physical signals may be produced. For example, in one embodiment, a material may be disposed within the potty training liner that becomes rough upon contact with a fluid. In such an embodiment, in addition to the wetness sensation, the user will also feel a tactile change touching their skin. The rough material may be caused to move along the skin due to fluid in contact with and acting on other parts of the liner, such as a swelling action. The tactile change, or roughness, felt by the user thereby informs the user that an incident has occurred if the wetness alone does not inform the user of the incident. Those of ordinary skill in the art will appreciate that other types of physical signals may be produced along with a rough sensation and wetting. Additionally, a physical signal may be used separately from a wetness signal. For example, as fluid is released from the user, wetness felt by the user will occur. However, in an instance where the fluid was absorbed too quickly to cause the user to notice, a corresponding physical change could result in informing the user of the incident independently from the wetness signal.

In still other embodiments, a vibratory or auditory signal may be used to inform a user of an incident. In such an embodiment, one or more substances, as discussed above, may be disposed within potty training liners. Upon contact with a fluid released by the user, the one or more substances may react to produce an uncomfortable motion that informs the user an incident has occurred. In other embodiments, upon contact with the fluid, the one or more substances may react to produce an auditory signal, such as a humming or hissing noise. Examples of substances that may be used to produce vibratory or auditory signals may be substances that turn gaseous when contacted by a water-based fluid, such as urine. Vibratory and auditory signals may be used along with wetness signals, tactile signals, and/or physical signals in order to inform a user an incident has occurred.

The initial sensory signal, or sensation, produced to the user will typically be wetness, as embodiments of the present disclosure are configured to create increased and/or prolonged contact between a user and a user released fluid. The additional above identified sensory signals may generally be referred to as secondary sensory signals. While the initial sensory signal will usually be present, in certain embodiments, the signal may not be strong enough or may not produce enough of a signal to inform the user of an incident. In such embodiments, secondary signals, or sensations, such as other tactile sensations, loss of heat sensations, vibratory sensations, or auditory sensations may also occur. Those of ordinary skill in the art will further understand that in certain embodiments, only secondary signals, sensations, may be used. Thus, in certain embodiments, the fluid may not be intended to produce a sensory signal, rather, the fluid may be intended to trigger a different tactile, vibratory, and/or auditory response.

In still other embodiments, changes to the physical structure of the potty training liners may result in a sensory signal. For example, when fluid is released by the user into the potty training liner, the fluid initially pools or otherwise wets a hydrophilic sensory strip. Over time, the fluid is absorbed into the core, as described in detail above. As the fluid is absorbed into the core, the superabsorbent material swells, thereby increasing in size. As the core swells, the potty training liners tends to sag down the legs of the user. In certain embodiments, the core and/or the potty training liner in general may be configured to at least double in size, thereby alerting the user due to the increased bulk of the potty training liner. Additionally, in certain embodiments, the physical structure of the potty training liner may be changed by elongating the potty training liner along the body of the user. During normal activity, the user may feel the swelling or sagging of the potty training liner, thereby producing an additional secondary sensory signal to the user.

As explained above, in certain embodiments, a hydrophilic sensory strip may not be used. In such an embodiment, a sensory portion of a potty training liner may provide the sensory signal or sensation to the user. Examples of sensory signals provided by sensory portion may include tactile, wetness, auditory, vibratory, etc. signals.

Although several embodiments and alternative implementations have been described, many other modifications and implementation techniques will be apparent to those skilled in the art upon reading this disclosure. Materials described as water-permeable may incorporate water-resistant components, but with an overall permeability. Likewise, materials described as waterproof may be water-resistant, and additionally allow some water to pass with extracted exposure times. Dimensions shown on Figures are suggested for one size embodiment, with other dimensions appropriate for other applications.

Although the specification may refer to “an”, “one”, “another”, or “some” embodiment(s) in several locations, this does not necessarily mean that each such reference is to the same embodiment(s), or that the feature only applies to a single embodiment.

Advantageously, embodiments of the present disclosure may provide an active training aid for potty training users. Potty training liners according to embodiments of the present disclosure may provide sensory signals, sensory sensations, to the user, thereby informing the user that an incident has occurred.

Also advantageously, embodiments of the present disclosure may provide more effective and faster training. Because the user experiences one or more sensory signals, sensory sensations, the user may experience a level of discomfort. The discomfort may thus inform a user that an incident has occurred. After repeated discomfort from incidents, the user may thus be more inclined not to incur an incident.

Also advantageously, embodiments of the present disclosure may more effectively potty train as the user experiences similar feedback as if they were wearing undergarments without a diaper. Because the potty training liners disclosed herein are configured to use released fluid, urine, to contact the user, rather than quickly remove the fluid from contact with the user, the sudden increase in size of the liner may cause the user to feel uncomfortable and prompt the user to take action. This is an advantage to trainers who may be resistant to the wetness signal alone and a further advantage to training in underwear without a liner. The increase in size and temporal contact between the wet potty training liner and the user may thus produce a sensory signal, sensory sensation, which causes discomfort for the user.

Also advantageously, embodiments of the present disclosure may decrease the amount of raw materials used in potty training. Diapers and training pants may use greater amounts of raw materials due to the size and intent of the products, i.e., to keep users feeling dry. Potty training liners according to embodiments disclosed herein are respectively smaller, thereby using fewer raw materials. The decrease in raw materials used may decrease the cost of potty training as well as ease the burden on landfills.

Also advantageously, embodiments of the present disclosure may provide a psychological benefit to users and parents during potty training. In the case of children, because they are wearing normal undergarments with a liner, instead of a traditional diaper or training pants, they may feel older and thus want to act older. For parents, embodiments encourage earlier training as a convenient and more effective aid for training. Earlier training avoids the development of resistance by willful trainers who are older due to delayed start of the training process and who have developed an entrenched habit of using their diaper rather than a toilet. Such psychological benefits may further decrease the training time, further decreasing the costs associated therewith.

Also advantageously, embodiments of the present disclosure may provide secondary sensory signals, secondary sensory sensations, that further speed potty training. Examples of secondary sensory signals may include, tactile, physical, loss of heat, vibration, auditory, and change in liner feeling. Moreover, the combination of initial sensory signals and secondary sensory signals may collectively speed potty training.

Also advantageously, embodiments of the present disclosure providing secondary sensory signals may increase the speed of potty training by providing a night time training aid. Secondary sensory signals may be optimized to provide a signal to a user that causes the user to wake up. As the user becomes accustomed to the discomfort of the secondary sensation, the user may wake up at night, further speeding the potty training process.

While the present invention has been described with respect to the above-noted embodiments, those skilled in the art, having the benefit of this disclosure, will recognize that other embodiments may be devised that are within the scope of the invention as disclosed herein. Accordingly, the scope of the invention should be limited only by the appended claims.

What is claimed is:

1. A potty training apparatus comprising:
a hydrophobic bottom layer;
a core disposed on the hydrophobic bottom layer;
a top layer comprising an acquisition layer disposed above the core; and
a hydrophilic sensory strip disposed on the top layer.

2. The potty training apparatus of claim 1, further comprising standing side gathers connected to at least one of the hydrophobic bottom layer and the top layer.

3. The potty training apparatus of claim 1, wherein hydrophilic sensory strip comprises cotton.

4. The potty training apparatus of claim 1, wherein the core comprises a superabsorbent material.

5. The potty training apparatus of claim 1, wherein the core is substantially flat.

6. The potty training apparatus of claim 1, wherein the core is substantially arcuate.

7. The potty training apparatus of claim 1, wherein the core comprises inward sloping sidewalk.

8. The potty training apparatus of claim 1, wherein the hydrophilic sensory strip is connected to the top layer with a waterproof adhesive.

9. The potty training apparatus of claim 1, wherein the acquisition layer is configured to prevent rapid absorption of fluids.

10. The potty training apparatus of claim 1, further comprising sensory locations disposed on the top layer.

11. The potty training apparatus of claim 1, wherein the hydrophilic sensory strip stays wet for at least 15 minutes.

12. A method of potty training a user comprising:
releasing a fluid comprising urine into a potty training liner, the potty training liner comprising a sensory section, wherein releasing the fluid wets the sensory section;
contacting the wet sensory section against the user; and
producing a sensory signal to the user.

13. The method of claim 12, further comprising holding the fluid above a core and in contact with the user.

14. The method of claim 12, wherein the sensory signal is a wetness.

15. The method of claim 12, further comprising increasing at least one of a length, a height, and a width of the potty training liner along the user.

16. The method of claim 15, further comprising producing a second sensory signal to the user by elongating the potty training liner.

17. The method of claim 12, further comprising contacting the fluid with a secondary sensory strip.

18. The method of claim 17, wherein the secondary sensory strip provides at least one of a vibratory, a tactile signal, and an auditory signal to the user.

19. The method of claim 12, further comprising contacting the fluid with a secondary sensory location disposed on the potty training liner.

20. A method of potty training a user, the method comprising:
providing a potty training liner in an unworn condition, wherein the potty training liner in the unworn condition comprises a central void region and a broad central channel; and
applying the potty training liner to the user, wherein the potty training liner in a worn condition comprises a closed central void region and a secondary void region having a pooling area for fluids released by the user.
